(19) United States
(12) Patent Application Publication
(10) Pub. No.: US 2025/0281625 A1
(43) Pub. Date: Sep. 11, 2025

(54) FOLIC ACID-CONJUGATED AMINOCLAY BASED NANOCOMPOSITES FOR THE SITE-SPECIFIC DRUG DELIVERY, METHOD FOR THEIR PREPARATION AND THEIR USE

(71) Applicant: Dongguk University Industry-Academic Cooperation Foundation, Seoul (KR)

(72) Inventors: Hyo Kyung HAN, Seoul (KR); Sang Hoon LEE, Jeju-si, Jeju-do (KR)

(73) Assignee: Dongguk University Industry-Academic Cooperation Foundation, Seoul (KR)

(21) Appl. No.: 18/289,798

(22) PCT Filed: May 6, 2022
(86) PCT No.: PCT/KR2022/006525

§ 371 Date: Jun. 10, 2024

(30) Foreign Application Priority Data
May 8, 2021 (KR) 10-2021-0059618

(51) Int. Cl. A61K 47/55; A61K 9/00; A61K 47/52

(52) U.S. Cl. CPC A61K 47/551; A61K 9/0053; A61K 47/52

(57) ABSTRACT

The present disclosure relates to a folic acid-conjugated aminoclay (FA-AC) nanocomposite for site-specific drug delivery, preparation methods thereof, and uses thereof. According to the present disclosure, a FA-AC binds to a drug to form a nanocomposite to increase site-specificity. Furthermore, the stability of the loaded drug in the gastrointestinal tract is improved by surface-coating a pH-sensitive polymer on the nanocomposite, which can significantly improve the therapeutic effect of the drug. Nanocomposites according to the present disclosure target folate receptors and can therefore be applied as effective drug carriers for the prevention and treatment of inflammatory disease or cancer in which folate receptors are overexpressed.

### TECHNICAL FIELD

The present disclosure relates to a folic acid-conjugated aminoclay (FA-AC) based nanocomposite for site-specific drug delivery, a preparation method thereof, and use thereof.

### BACKGROUND ART

Site-specific drug delivery systems are very effective in reducing drug side effects and increasing treatment effectiveness by selectively delivering drugs only to specific organs or tissues. To this end, research is being actively conducted to facilitate site-specific drug delivery and internalization by binding, to a drug carrier, a ligand that can selectively interact with a specific receptor on the target cell surface.

Among targeting ligands, folic acid plays an essential role in cell survival and has high binding affinity to folate receptors, which are a biomarker for cancer and inflammatory diseases. Folate receptors are rarely expressed in normal cells, but are overexpressed in monocytes, macrophages activated in inflammatory diseases, and various malignant tumor cells, including ovarian cancer. Therefore, folic acid-conjugated nanoparticles have attracted significant attention for selective drug delivery to disease sites in anti-cancer treatments and inflammatory disease treatments. In particular, the development of a drug delivery system targeting the folate receptor, which is overexpressed in malignant tumor cells compared to normal cells, has long received attention for the development of site-specific anticancer drugs. In recent years, clinical trials of monoclonal antibody drugs targeting the folate receptor as well as small molecule drugs have been actively conducted.

Additionally, studies are being conducted to increase target cell selectivity of drug delivery in the treatment of inflammatory diseases. For example, Zhang et al. fabricated PEG-folic acid functionalized PLGA/PLA (poly lactic-co-glycolic acid/poly lactic acid) nanoparticles loaded with 6-shogaol for attenuation of ulcerative colitis (Zhang et al., J. Crohn's Colitis, 2018, 12, 217-229). These folic acid-conjugated nanocarriers may be useful for selective drug delivery to activated macrophages at the site of inflammation. Ulcerative colitis, one of the inflammatory diseases, is a chronic disease that causes inflammation and ulcers in the innermost lining of the large intestine. Ulcerative colitis is a chronic disease that causes symptoms such as abdominal pain, bloody diarrhea, fatigue, fever, and weight loss, and in most cases, symptoms are repeatedly worsen and improved, seriously affecting the quality of life. In addition, chronic inflammation increases the risk of colon cancer.

In patients with ulcerative colitis, large numbers of monocytes migrate to the intestines, activating macrophages and thus inducing the secretion of inflammatory cytokines such as tumor necrosis factor-α (TNF-α), interleukin-6 (IL-6), and interleukin-23 (IL-23). Infliximab (IFX), a chimeric monoclonal antibody, binds to TNF-α and reduces inflammation caused by TNF-α, and is therefore used as a treatment for ulcerative colitis. However, because IFX is administered by intravenous or subcutaneous injection, the patient compliance is low and systemic immunosuppression occurs. Therefore, there is a need to develop a drug delivery system that can increase patient compliance and selectivity of the inflammatory site.

Meanwhile, aminoclay, which is a metal phyllosilicate (layered silicate) to which a 3-aminopropyl group is introduced, is water-soluble and has a positive charge when dispersed in water. Aminoclay exhibits many favorable properties as a drug carrier including the favorable safety profiles and ability to enhance the bioavailability of macromolecules. In some embodiments, aminoclay has a layered structure, which allows drugs to be enclosed between layers or adsorbed on a large particle surface. Aminoclay can be used to prepare a variety of nanocomposites through spontaneous self-assembly by electrostatic attraction with macromolecules such as small molecule drugs, proteins, viruses and polymers. Aminoclay has a large surface area, allowing various surface modifications with targeting ligands via electrostatic interaction or conjugation. In addition, as aminoclay is non-toxic and is rapidly excreted from the body through urine and feces, there is a low risk of long-term tissue accumulation (Yang et al., J Materials Chemistry B., 2014, 2, 7567-7574).

### DISCLOSURE

### Technical Problem

The inventors of the present application synthesized folic acid-conjugated aminoclay (FA-AC), in which folic acid and aminoclay bind to each other, and bound the same to biopharmaceuticals such as protein drugs or antibody therapeutic agents to form a nanocomposite, and found that the nanocomposite is not only effective in selectively delivering drugs, but also maintains the structural stability of the biopharmaceutical loaded within the nanocomposite, thereby completing the present disclosure.

Accordingly, one objective of the present disclosure is to provide a FA-AC based nanocomposite comprising FA-AC, in which folic acid and aminoclay bind to each other, and a drug binding thereto.

Another objective of the present disclosure is to provide a composition for drug delivery, comprising the FA-AC based nanocomposite and a pharmaceutically acceptable carrier.

Another objective of the present disclosure is to provide a pharmaceutical composition for the prevention or treatment of inflammatory diseases or cancer, comprising the FA-AC based nanocomposite and a pharmaceutically acceptable carrier.

Another objective of the present disclosure is to provide a pH-sensitive FA-AC based nanocomposite in which a pH-sensitive polymer is coated on the FA-AC based nanocomposite.

Another objective of the present disclosure is to provide a pharmaceutical composition for oral administration comprising the pH-sensitive FA-AC based nanocomposite and a pharmaceutically acceptable carrier.

Another objective of the present disclosure is to provide a method of preparing a FA-AC based nanocomposite, comprising (a) binding folic acid and aminoclay to each other to prepare a folic acid-conjugated aminoclay (FA-AC); and (b) binding a drug to the FA-AC to prepare a FA-AC based nanocomposite.

### Technical Solution

In order to achieve these objectives, the present disclosure provides a folic acid-conjugated aminoclay (FA-AC) based nanocomposite comprising FA-AC, in which folic acid and aminoclay bind to each other, and a drug bound thereto.

In the present disclosure, FA-AC was confirmed to maintain the planar layered structure of aminoclay and to have no intracellular toxicity. Additionally, while folic acid has very low water solubility, FA-AC according to the present disclosure is soluble in water in an amount of up to 10 mg/mL.

The FA-AC based nanocomposite according to the present disclosure targets a folate receptor. Folate receptors are rarely expressed in normal cells, but are overexpressed in a variety of cancer cells and inflammatory cells. Therefore, FA-AC based nanocomposites according to the present disclosure can be applied to drugs used for the prevention or treatment of various cancer cells and inflammatory diseases to improve their prevention or treatment effects for various cancer cells and inflammatory diseases.

For example, a nanocomposite prepared by binding an antibody therapeutic agent, such as infliximab, and FA-AC to each other can improve inflammation treatment effects by acting selectively on inflammatory cells overexpressing folate receptors while keeping the structure of infliximab stable.

According to the present disclosure, aminoclay is a metal phyllosilicate into which a 3-aminopropyl group is introduced, and disperses well in water and has a positive charge. In some embodiments, the metal may be magnesium (Mg), but is not limited thereto. The aminoclay is a cationic nanosheet and may interact electrostatically with anionic molecules. For example, aminoclay may be 3-aminopropyl functionalized magnesium phyllosilicate, but is not limited thereto, and magnesium in the layered structure may be substituted with other cations including calcium, iron, aluminum, manganese, zinc, etc.

According to the present disclosure, drugs that may form a nanocomposite with FA-AC may be protein drugs, peptide drugs, DNA, RNA, antibody therapeutic agents, immunotherapy agents, or small molecule drugs, but are not limited thereto. Protein drugs, peptide drugs, DNA, RNA, antibody therapeutic agents, immunotherapy agents, or small molecule drugs may be any protein, peptide, DNA, RNA, antibody, immunotherapy agents or small molecule drugs that are suitable for use as a drug.

The protein drugs are proteins for drugs produced based on genetic recombination, cell culture, or bio-processing, and may include protein drugs used for disease treatment, etc. through mass production using microorganisms or animal cell systems. For example, the protein drugs may be a linear protein drug or a cyclic protein drug. The protein drugs may also be a modified or derivatized protein drug, such as a fatty acid acylated protein drug or a fatty discrete acylated protein drug. In some embodiments, the protein drugs include one or more selected from the group consisting of albumin, lilaglutide (Lira), teriparatide, insulin, insulin analogs, glucagon-like peptide-1 (GLP-1), GLP-2, semaglutide, exenatide, exendin-4, lixisenatide, taspoglutide, albiglutide, dulaglutide, oxyntomodulin, amylin, somatostatin analogs, goserelin, buserelin, leptin, glatiramer acetate, leuprolide, osteocalcin, human growth hormone (hGH), glycopeptide antibiotic, bortezomib, cosyntropin, Menotropins, gonadotropin releasing hormone (GnRH), somatropin, calcitonin, oxytocin, lepirudin, carfilzomib, icatibant, and aldesleukin, but are not limited thereto.

The antibody therapeutic agents refer to a monoclonal antibody, a polyclonal antibody, and a protein containing a monoclonal antibody fragment or a polyclonal antibody fragment, which can bind specifically to an antigen associated with a specific disease. For example, antibody therapeutic agents may be trastuzumab, abciximab, rituximab, basiliximab, cetuximab, alemtuzumab, bevacizumab, pavilizumab, adalimumab, certolizumab, eculizumab, catumaxomab, golimumab, efalizumab, lorvotuzumab, brentuximab, glembatumumab, Ipilimumab, Nivolumab, Pembrolizumab, Atezolizumab, Avelumab, Durvalumab, Cemiplimab, or Infliximab, but are not limited thereto. In some embodiments, the antibody therapeutic agents may be Infliximab.

The immunotherapy agents are a material that allows immune cells to attack cancer cells more effectively by suppressing cancer cells from evading the immune response or activating the immune response, and include immunotherapy agents that are widely known in the art. For example, immunotherapy agents include anti-PD1, anti-PDL1, anti-CTLA4, anti-LAG3, anti-VISTA, anti-BTLA, anti-TIM3, anti-HVEM, anti-CD27, anti-CD137, anti-OX40, anti-CD28, anti-PDL2, anti-GITR, anti-ICOS, anti-SIRPα, anti-ILT2, anti-ILT3, anti-ILT4, anti-ILT5, anti-EGFR, anti-CD19, anti-TIGIT, etc. and are not limited thereto.

The small molecule drugs refer to a biologically active compound (or a salt thereof) that can produce a desired, beneficial, and/or pharmacological effects in a subject, and examples thereof are methotrexate, irinotecan, topotecan, sorafenib, doxorubicin, prednisolone, etc., but are not limited thereto.

Additionally, the present disclosure provides a composition for drug delivery, comprising the FA-AC based nanocomposite and a pharmaceutically acceptable carrier.

The composition for drug delivery according to the present disclosure may be administered through various administration routes suitable for drugs, such as oral administration, injection (subcutaneous or intramuscular injection), buccal administration, nasal administration, sublingual administration, lung administration, or dermal administration.

The composition for drug delivery may be formulated, according to conventional methods, in various forms including: oral dosage forms, such as, powders, granules, tablets, capsules, suspensions, emulsions, syrups, aerosols, oral patches; external preparations; external patches; and sterilized injection solutions.

The composition for drug delivery may include one or more pharmaceutically acceptable carriers in addition to the FA-AC based nanocomposite. In this regard, the pharmaceutically acceptable carriers refer to excipients and diluents which are commonly used in formulations, and examples thereof are lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, etc. and are not limited thereto. In addition to these ingredients, lubricants, wetting agents, sweeteners, flavoring agents, emulsifiers, suspending agents, preservatives, etc. may be further included as the pharmaceutically acceptable carriers.

Additionally, the present disclosure provides a pharmaceutical composition for the prevention or treatment of inflammatory diseases or cancer, comprising the FA-AC based nanocomposite and a pharmaceutically acceptable carrier.

The FA-AC based nanocomposite of the present disclosure targets a folate receptor. Therefore, the pharmaceutical composition comprising the FA-AC based nanocomposite and a pharmaceutically acceptable carrier exhibits site-specificity for inflammatory cells and cancer cells overexpressing folate receptors, and thus shows excellent effects for the prevention or treatment of inflammatory diseases or cancer.

The “inflammatory diseases” that can be prevented or treated by using the pharmaceutical composition comprising the FA-AC based nanocomposite and a pharmaceutically acceptable carrier according to the present disclosure include, for example, inflammatory bowel diseases such as ulcerative colitis, Crohn's disease, Behcet's disease, etc. and is not limited thereto. In some embodiments, the inflammatory diseases may be ulcerative colitis.

The “cancer” that can be prevented or treated by using the pharmaceutical composition comprising the FA-AC based nanocomposite and a pharmaceutically acceptable carrier according to the present disclosure refers to a disease in which normal tissue cells proliferate unrestrictedly due to any cause and continue to develop rapidly regardless of the life phenomena of the organism or the surrounding tissue conditions. The cancer includes, but is not limited to, dysplastic, hyperplastic, solid tumor and hematopoietic stem cell cancer, and includes various cancer types known in the art. In addition, cancer may include cancer that occurs in various organs, for example, brain cancer, heart cancer, lung cancer, stomach cancer, liver cancer, colon cancer, kidney cancer, pancreas cancer, colon cancer, prostate cancer, liver cancer, bone cancer, nervous system cancer, blood cancer, skin cancer, and thyroid cancer, breast cancer, uterine cancer, cervical cancer, ovarian cancer, bladder cancer, adrenal cancer, etc., but is not limited thereto. Other types of cancer cells include gliomas (schwannoma, glioblastoma, astrocytoma), neuroblastoma, pheochromocytoma, paraganglioma, meningioma, adrenocortical cancer, medulloblastoma, rhabdomyosarcoma, various types of vascular cancer, osteoblastic osteocarcinoma, uterine fibroids, salivary gland cancer, choroid plexus cancer, and megakaryocytic leukemia; and skin cancer including sarcomas and melanoma, for example, malignant melanoma, basal cell carcinoma, squamous cell carcinoma, Karposi's sarcoma, moles dysplastic nevi, lipoma, hemangioma, dermatofibroma, keloid, fibrosarcoma, or angiosarcoma.

The term “prevention” as used in the present disclosure refers to any act by which the occurrence, spread and recurrence of a disease is hindered or delayed by the administration of a pharmaceutical composition according to the present disclosure, and the term “treatment” as used herein refers to any act by which the symptoms of a disease are ameliorated or beneficially altered by the administration of a pharmaceutical composition according to the present disclosure.

The present disclosure provides use of the FA-AC based nanocomposite for the prevention or treatment of inflammatory diseases or cancer.

The present disclosure provides use of the FA-AC based nanocomposite for the production of a drug for the prevention or treatment of inflammatory diseases or cancer.

The present disclosure also provides a method for the prevention or treatment of inflammatory diseases or cancer, comprising administering the FA-AC based nanocomposite to a subject in need.

The pharmaceutical composition according to the present disclosure may be administered orally or parenterally (e.g., intravenously, subcutaneously, intraperitoneally, or topically) depending on the desired method, and the dosage thereof may vary depending on the patient's condition and weight, and the degree of the disease, the drug form, the administration route, and time, and may be appropriately selected by a person skilled in the art.

The pharmaceutical composition according to the present disclosure may be administered in a pharmaceutically effective amount. The term “pharmaceutically effective amount” as used in the present disclosure refers to an amount sufficient to treat a disease with a reasonable benefit/risk ratio applicable to medical treatment, and the effective dose level may be determined according to the type and severity of the patient's disease, the activity of the drug, the sensitivity to the drug, the time of administration, the route of administration and the rate of elimination, the duration of treatment, factors including concomitant drugs, and other factors well known in the medical field.

The pharmaceutical composition according to the present disclosure may be administered as an individual therapeutic agent or in combination with other therapeutic agents, and may be administered sequentially or simultaneously with conventional therapeutic agents, and may be administered in single or multiple doses. Considering all of these factors, it is important to administer an amount that can achieve the maximum effect with the minimum amount without side effects, which may be easily determined by a person skilled in the art.

In some embodiments, the effective amount of the pharmaceutical composition according to the present disclosure may vary depending on the patient's age, gender, condition, body weight, route of administration, the absorption, inactivation rate and excretion rate of an active ingredient in the body, type of disease, and concomitant drug.

Additionally, the present disclosure provides a method for the prevention or treatment of inflammatory diseases or cancer, comprising administering the pharmaceutical composition to a subject. The term “subject” as used herein refers to a subject in need of treatment for a disease and, more specifically, to a mammal, such as a human or non-human primate, mouse, dog, cat, horse, and COW.

Furthermore, the present disclosure provides a pH-sensitive FA-AC based nanocomposite comprising FA-AC, in which folic acid and aminoclay bind to each other, and a drug bound thereto, in which a pH-sensitive polymer is coated on the FA-AC based nanocomposite.

In the present disclosure, since a pH-sensitive polymer is coated on the FA-AC based nanocomposite, stability in the gastrointestinal tract can be improved and premature release of the drug can be prevented. In the present disclosure, it was confirmed that the structural stability of the loaded drug was well maintained in a nanocomposite which is coated with a pH-sensitive polymer after the drug and FA-AC bind to each other.

The pH-sensitive polymer in the present disclosure is a polymer for preventing a drug from being dissolved or released by stomach acid before reaching the intestine, and may be, but is not limited to, poly(methacrylic acid-co-methyl acrylate) copolymers. Poly(methacrylic acid-co-methyl acrylate) copolymers are used for pH-dependent drug release in the gastrointestinal tract, and may be the Eudragit family of polymers, which are copolymers of acrylate and methacrylate. Eudragit series exhibits variable pH-dependent solubility depending on the ratio of polymerization materials and is very stable against hydrolysis. Eudragit®S has a carboxyl group in the molecule that can be ionized, resulting in an insoluble and stable coating layer at low pH, but at pH above 7, the carboxyl group ionizes, causing the coating layer to dissolve and the contents to be released. In some embodiments, the poly(methacrylic acid-co-methyl acrylate) copolymer may be Eudragit® S100.

According to the present disclosure, when a pH-sensitive FA-AC based nanocomposite (Eudragit® S100/folic acid-conjugated aminoclay-infliximab: EFA-AC-IFX) in which Eudragit® S100 is coated on the composite of infliximab and FA-AC was orally administered to colitis-induced mice, it was confirmed that there was less weight loss compared to the normal control group and that TNF-α concentration was similar to that of the normal control group. These experimental results show that coating the FA-AC based nanocomposite with a pH-sensitive polymer significantly improves the stability in the gastrointestinal tract and the therapeutic effect upon oral administration.

Accordingly, the present disclosure provides a pharmaceutical composition for oral administration comprising a pH-sensitive FA-AC based nanocomposite and a pharmaceutically acceptable carrier. The pharmaceutical composition for oral administration may be used in the form of a general pharmaceutical preparation. For example, the composition according to the present disclosure may be formulated in the form of preparations for oral administration such as tablets, granules, capsules, suspensions, etc., and these preparations may be prepared using conventional pharmaceutically acceptable carriers such as excipients, binders, disintegrants, lubricants, solubilisers, colourants, coatings, suspending agents, and preservatives. Additionally, the administered dose of the pharmaceutical composition for oral administration may be determined by an expert according to various factors such as the patient's condition, age, gender, and complications. Additionally, the pharmaceutical composition for oral administration may further include a pharmaceutically acceptable additive in addition to the nanocomposite.

The detailed description of the pharmaceutical composition is as described above.

The present disclosure provides use of the pH-sensitive FA-AC based nanocomposite for the prevention or treatment of inflammatory diseases or cancer.

The present disclosure provides use of the pH-sensitive FA-AC based nanocomposite for the production of a drug for the prevention or treatment of inflammatory diseases or cancer.

The present disclosure also provides a method for the prevention or treatment of inflammatory diseases or cancer, comprising administering the pH-sensitive FA-AC based nanocomposite to a subject in need.

The present disclosure also provides a method of preparing a FA-AC based nanocomposite, comprising (a) binding folic acid and an aminoclay to each other to prepare a FA-AC; and (b) binding a drug to the folic acid-conjugated aminoclay to prepare a FA-AC based nanocomposite.

Regarding the preparation method of the present disclosure, the aminoclay may be a metal phyllosilicate into which a 3-aminopropyl group is introduced, for example, 3-aminopropyl functionalized magnesium phyllosilicate, but is not limited thereto.

The drug may be a protein drug, a peptide drug, DNA, RNA, an antibody therapeutic agent, an immunotherapy agent, or a small molecule drug, but is not limited thereto. Descriptions of the protein drugs, peptide drugs, DNA, RNA, antibody therapeutic agents, immunotherapy agents, and small molecule drugs are as described above.

In an embodiment, the method of preparing the FA-AC based nanocomposite of the present disclosure may further include (c) coating a pH-sensitive polymer on the FA-AC based nanocomposite.

Regarding the present disclosure, since a pH-sensitive polymer is coated on the FA-AC based nanocomposite, stability in the gastrointestinal tract can be improved and premature release of the drug can be prevented.

The pH-sensitive polymer may be a poly(methacrylic acid-co-methyl acrylate) copolymer, for example, Eudragit®S100, but is not limited thereto.

### Advantageous Effects of Disclosure

A nanocomposite of a drug and folic acid-conjugated aminoclay (FA-AC based nanocomposite) according to the present disclosure is selectively delivered to cells overexpressing folate receptors, showing excellent site-specificity, low cytotoxicity, and structural stability of the loaded drug, thereby significantly improving the therapeutic effect of the drug. Therefore, the nanocomposite of the present disclosure can be usefully used as a drug carrier effective for the prevention and treatment of inflammatory disease or cancer in which folate receptors are overexpressed. In addition, when a pH-sensitive polymer (e.g., Eudragit® S100) is coated on the FA-AC based nanocomposite, premature release of the drug in the stomach and upper small intestine can be prevented, thereby providing orally administered formulations with both improved stability of the drug in the gastrointestinal track and site-specificity.

### BRIEF DESCRIPTION OF DRAWINGS

FIG. 1A shows the results of X-ray powder diffraction analysis of folic acid-conjugated aminoclay (FA-AC) and FIG. 1B shows a transmission electron microscope (TEM) photograph of FA-AC.

FIG. 2 shows the results of evaluating the cytotoxicity of FA-AC in RAW 264.7 cells and Caco-2 cells.

FIG. 3 shows the circular dichroism (CD) spectra of (i) pure infliximab (IFX) in phosphate-buffer saline (PBS), (ii) nanocomposite of infliximab and FA-AC (FA-AC-IFX) in PBS, and (iii) Eudrazit® S100-coated FA-AC-IFX (EFA-AC-IFX) in PBS.

FIG. 4 shows a photograph showing cellular uptake, in macrophages, of nanoparticles loaded with fluorescent labeled bovine serum albumin (FITC-BSA).

FIG. 5A shows the graph of body weight change and FIG. 5B shows the graph of TNF-α concentration change, after oral administration of EFA-AC-IFX in colitis-induced mice to evaluate the in vivo efficacy of EFA-AC-IFX.

### MODE FOR INVENTION

Hereinafter, the present disclosure will be described in more detail through examples. However, these examples are for illustrative purposes only, and the scope of the present disclosure is not limited by these examples.

### Example 1. Preparation of Folic Acid-conjugated Aminoclay (FA-AC)

FA-AC was prepared to make nanoparticles to target folate receptors overexpressed in tumor cells and inflammatory cells. Folic acid (50 mg, 113.2 μmol) was dissolved in a mixture containing ethanol and dimethyl sulfoxide (DMSO) (3:1 v/v, 100 ml), and then added to 100 ml of MgCl₂·6H₂O (8.4 g, 41.3 mmol) ethanol solution. While stirring the obtained mixed solution at 250 rpm, 3-aminopropyltriethoxysilane (APTES) (13 ml, 5.85 mmol) was added dropwise thereto. Stirring was maintained overnight to ensure sufficient FA-AC formation. The resulting precipitate was centrifuged, washed five times with ethanol, and dried at 40° C.

### Example 2. Characteristics of FA-AC

The particle size and zeta potential of FA-AC were measured by dynamic light scattering (DLS) using a Zetasizer Nano-ZS90 (Malvern Instruments, Malvern, UK). The structural properties of FA-AC were analyzed using X-ray powder diffraction (XRPD) technique. The XRPD pattern was confirmed at room temperature using an X-ray diffractometer (X'Pert APD, PHILIPS, Amsterdam, Netherlands). Morphological characteristics of FA-AC were monitored by transmission electron microscopy (TEM) (JEM-2100F, JEOL Ltd., Tokyo, Japan). The cytotoxicity of FA-AC in RAW 264.7 cells and Caco-2 cells was confirmed through MTT analysis. Cytotoxicity was measured 48 hours after culture.

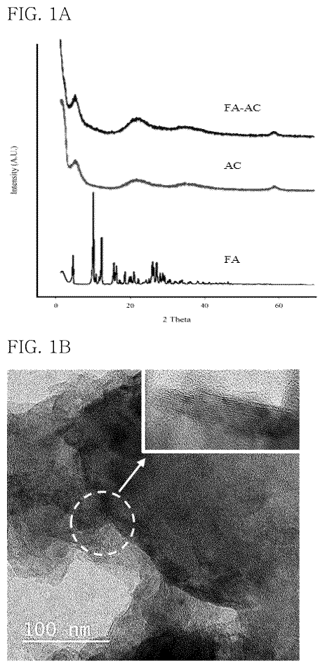

Through X-ray powder diffraction analysis and transmission electron microscopy (TEM) observation, it was confirmed that the layered structure of aminoclay was maintained in FA-AC (FIG. 1). X-ray powder diffraction analysis and TEM analysis results suggest that the layered sheet structure is not modified even after folic acid binds to the aminopropyl group of aminoclay.

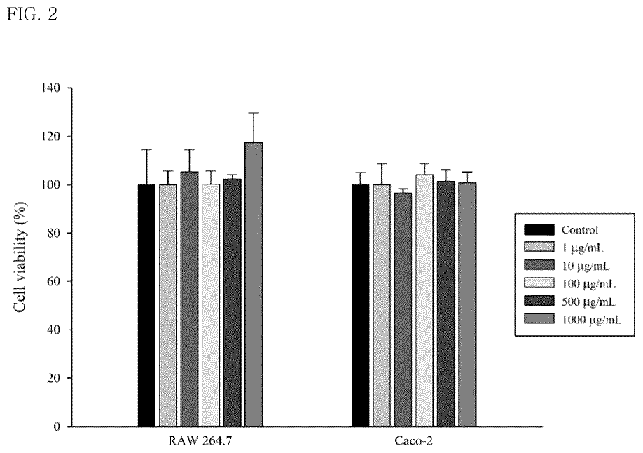

While folic acid has a very low water solubility (0.0016 mg/g), FA-AC was delaminated in water and dissolved in an amount of up to 10 mg/mL in water. The results of evaluating the cytotoxicity of FA-AC in RAW 264.7 cells and Caco-2 cells are shown in FIG. 2, and FA-AC was found to be non-cytotoxic in a concentration of up to 1 mg/mL in RAW 264.7 cells and Caco-2 cells.

### Example 3. Preparation of Infliximab-loaded Nanoparticles

The nanocomposite of infliximab and FA-AC (FA-AC-IFX) was prepared through electrostatic binding of positively charged FA-AC and negatively charged infliximab at room temperature. An infliximab solution (1 mg/mL) was added dropwise to the aqueous solution of FA-AC (4 mg/mL) and stirred for 3 hours. FA-AC-IFX (1 mg) obtained by centrifuging the resulting precipitate, was suspended in water (1 mL) and then added dropwise into 0.2% Eudragit® S100 ethanol solution (1 mL) to perform surface coating of FA-AC-IFX. After stirring for 30 minutes, the resulting Eudragit® S100 coated nanoparticles (EFA-AC-IFX) were centrifuged. FA-AC-IFX and EFA-AC-IFX were freeze-dried using 2% trehalose.

The nanocomposite (FA-AC-IFX) of infliximab (INF) and folic acid-conjugated aminoclay (FA-AC) exhibited a high drug entrapment efficiency of about 94% and an average particle size of 140±1.27. nm, and the zeta potential of 3.25±0.06 mV. In addition, EFA-AC-IFX in which Eudragit® S100 was coated on FA-AC-IFX to protect infliximab-loaded nanoparticles from the acidic environment of the stomach, had an average particle size of 384±10.3 nm and a high entrapment efficiency of 87% or more, and the zeta potential of −13.6±0.55 mV.

### Example 4. Structural Characterization of Infliximab-loaded Nanoparticles

The formations of FA-AC-IFX and EFA-AC-IFX were confirmed through various structural analyses. The Fourier-transform infrared spectroscopy (FT-IR) spectrum of FA-AC-IFX showed a strong absorbance band at 1652-1654 cm⁻¹ due to the α-helix region of infliximab; and the Si—C band at 1130 cm⁻¹, the Si—O—Si band at 1008 cm⁻¹, and the Mg—O—Si band at 559-497 cm⁻¹, due to the network layered silicate. The FT-IR spectrum of EFA-AC-IFX showed a carboxyl group at 1700 cm⁻¹ due to Eudragit® S100. Additionally, the shape of the nanoparticles identified by TMS was spherical.

Since changes in protein structure can affect the biological activity, immunogenicity, and toxicity of proteins, stabilization of protein structure is important for the formulation of protein pharmaceuticals. Therefore, the structural stability of infliximab loaded on the nanocomposite was investigated using circular dichroism (CD) spectroscopy.

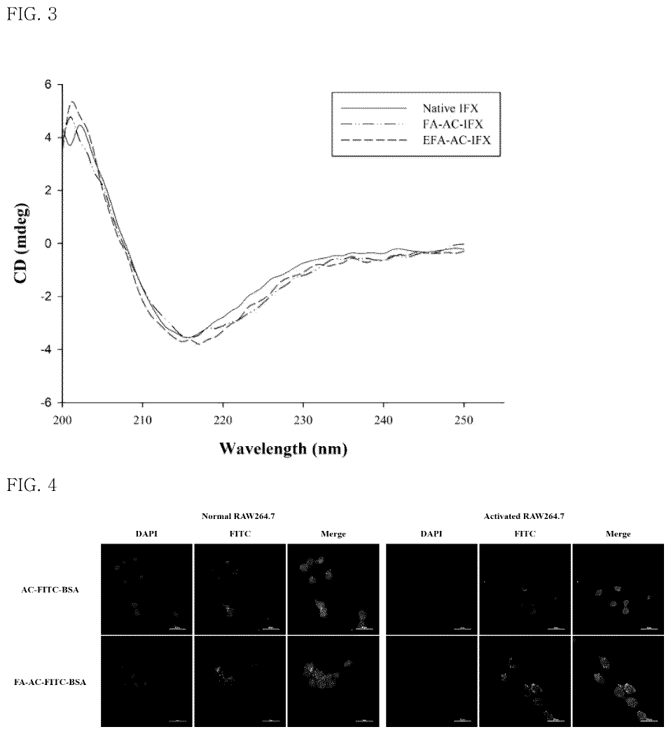

As can be seen in FIG. 3, the CD spectra of FA-AC-IFX and EFA-AC-IFX almost overlap with the CD spectrum of pure infliximab, which suggests that the structural stability of infliximab loaded on the nanocomposites is well maintained.

### Example 5. Drug Release Properties

Considering the pH conditions in the gastrointestinal tract, nanoparticles (corresponding to 0.2 mg/mL infliximab) were dispersed in pH 1.2 and pH 7.4 buffer solutions and stirred at 100 rpm at 37° C. Samples were collected at designated time points and the concentration of the released drug was analyzed by high-performance liquid chromatography (HPLC).

At pH 1.2, FA-AC-IFX rapidly released the drug and released more than 90% of the drug within 30 minutes. On the other hand, EFA-AC-IFX had a very low drug release of less than 5% at pH 1.2 because the outer coating layer, Eudragit® S100, was insoluble below pH 7.0. However, since the Eudragit® S100 coating layer dissolves at pH 7.4, the drug release from EFA-AC-IFX at pH 7.4 was increased by 67% over 24 hours. The pH-dependent drug release properties of EFA-AC-IFX show that it can reduce premature drug release in the stomach and upper small intestine and deliver more drug to the large intestine.

### Example 6. Site-specificity of FA-AC Based Nanoparticles

To evaluate the targeting ability of FA-AC based nanocomposites, cellular uptake studies of nanocomposites (FA-AC-FITC-BSA) loaded with fluorescent labeled bovine serum albumin (FITC-BSA) was examined in RAW 264.7 cells. Cells were spread in a 12-well plate at the population of of 3×10⁵ cells per well. To prepare activated RAW 264.7 cells, after 24 hours of culture, the medium was replaced with culture medium containing 100 ng/ml lipopolysaccharide (LPS). Two days after seeding, the medium was removed and the cells were washed twice with serum-free RPMI 1640 medium. Each agent (FA-AC-FITC-BSA and AC-FITC-BSA) was added to the control (unactivated RAW 264.7 cells) and activated RAW 264.7 cells at a concentration equivalent to 0.1 mg/mL FITC-BSA, followed by the culturing using serum-free RPMI 1640 medium at 37° C. for 1 hour. The drug solution was removed and the cells were washed three times with cold serum-free RMPI 1640 medium. After cell lysis, the cell lysate was centrifuged at 15,000×g for 5 minutes. After collecting the supernatant, the FITC-BSA concentration was measured using a fluorescence plate reader at the excitation wavelength of 488 nm and the emission wavelength of 520 nm.

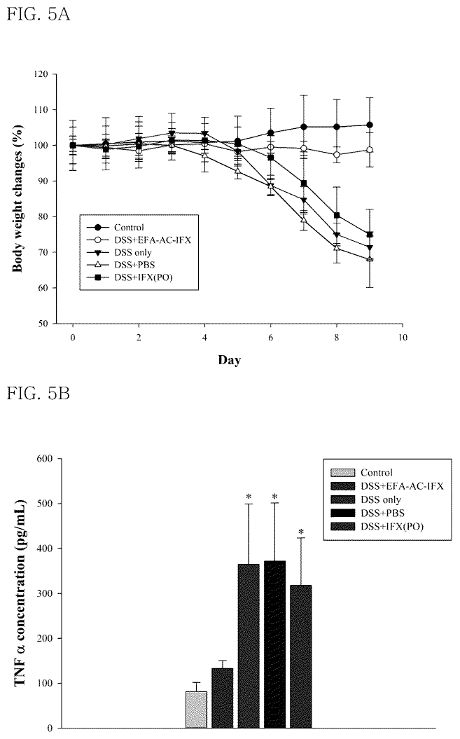

The FA-AC based nanocomposite showed a significantly higher cellular uptake in activated RAW 264.7 cells overexpressing folate receptors compared to control cells. In addition, the intracellular distribution of FA-AC-FITC-BSA was confirmed through bioimaging analysis using confocal laser scanning microscopy (CLSM). As shown in FIG. 4, after culturing activated RAW 264.7 cells with FA-AC-FITC-BSA, a much stronger FITC-BSA fluorescence intensity was detected in the cytoplasm of the cells surrounding the nucleus stained with 4′,6-diamidino-2-phenylindole (DAPI). These results show that FA-AC based nanoparticles can be effectively delivered to target cells overexpressing folate receptors.

### Example 7. In Vivo Efficacy Evaluation

Before inducing colitis, 8-week-old C57BL/6 mice were raised under standard conditions at 21° C. to 22° C. in a 12-hour light/dark cycle for 7 days. On the day of the experiment (Day 0), mice were randomly divided into five groups: a healthy control group and four experimental groups [dextran sodium sulfate administration group (DSS), dextran sodium sulfate and phosphate-buffer saline (PBS) administration group (DSS+PBS), dextran sodium sulfate and infliximab administration group (DSS+IFX), and dextran sodium sulfate and EFA-AC-IFX administration group (DSS+EFA-AC-IFX)]. Colitis was induced in mice by adding 2.0% (w/v) dextran sodium sulfate to drinking water.

To investigate the therapeutic effect of nanoparticles, colitis-induced mice in each experimental group were orally administered with 200 μL of PBS, infliximab (10 mg/kg), or EFA-AC-IFX (10 mg/kg) once daily for 8 days. On Day 8, drinking water containing dextran sodium sulfate was replaced with pure water. During the experiment, changes in body weight, stool consistency, and the presence of blood in the stool or anus were checked daily. Mice were sacrificed on day 9, and the colon was collected to evaluate the therapeutic efficacy of the drug-loaded nanocomposite.

### (1) Histological Analysis

To evaluate the therapeutic efficacy of EFA-AC-IFX for colitis, mouse colon tissues were histologically analyzed using hematoxylin-eosin (H&E) staining. The mouse colon sample was fixed in a PBS containing 4% paraformaldehyde and then embedded in wax. The embedded colon samples were then cut into 5-μm-thick sections using a microtome (Leica Biosystems), stained with H&E, and scanned using an Eclipse Ti-U inverted microscope (Nikon, Tokyo, Japan). The severity of colitis in each colonic section was assessed using a blinded approach by measuring mucosal features, such as epithelial damage, mucosal edema, and inflammatory cell infiltration of the mucosa.

Oral administration of EFA-AC-IFX to colitis-induced mice attenuated the progression of disease and was effective in reducing colitis-induced weight loss, tissue damage, and tumor necrosis factor-α (TNF-α) levels. As shown in FIG. 5A, orally administered EFA-AC-IFX minimized weight loss caused by colitis and maintained body weight similar to that of the healthy control group.

While the colon length of healthy mice (control group) was 10.3±0.58 cm, the induction of colitis by dextran sodium sulfate significantly reduced the colon length of mice to 6.7±0.29 cm. Even in the group administered orally with infliximab or PBS, the colon length of mice was significantly reduced (6.2±0.25 cm and 6.4±0.17 cm, respectively), confirming that oral administration of infliximab itself is not effective in treating colitis. On the other hand, the colon length of mice administered orally with EFA-AC-IFX was 9.6±0.10 cm, which was similar to that of healthy mice. Additionally, histological observations confirmed that EFA-AC-IFX was very effective in alleviating tissue damage caused by colitis.

### (2) Measurement of Levels of Inflammatory Cytokines

The level of TNF-α, one of the important inflammatory mediators involved in the pathogenesis of ulcerative colitis, is increased by inducing colitis in mice. Therefore, the concentration of TNF-α in colonic samples obtained from each experimental group was quantified using a TNF-α ELISA kit. As shown in FIG. 5B, when infliximab itself was orally administered, the concentration of TNF-α was significantly increased compared to the control group, whereas when EFA-AC-IFX was orally administered, the concentration of TNF-α was maintained at low levels similar to those of the control group. These results suggest that EFA-AC-IFX is an effective oral delivery system for infliximab, reducing the secretion of TNF-α caused by colitis and being effective in improving inflammation.

The description of the present disclosure described above is for illustrative purposes, and those skilled in the art would understand that the present disclosure can be easily modified into other specific forms without changing the technical concept or essential features of the present disclosure. Therefore, the test examples described above should be understood in all respects as illustrative and not restrictive.

1. Folic acid-conjugated aminoclay (FA-AC) based nanocomposite, comprising
FA-AC, in which folic acid and aminoclay bind to each other, and
a drug binding thereto.

2. The FA-AC based nanocomposite of claim 1, wherein aminoclay is a metal phyllosilicate to which a 3-aminopropyl group is introduced.

3. The FA-AC based nanocomposite of claim 1 or 2, wherein metal is magnesium (Mg).

4. The FA-AC based nanocomposite of any one of claims 1 to 3, wherein the drug is a protein drug, a peptide drug, DNA, RNA, an antibody therapeutic agent, an immunotherapy agent, or a small molecule drug.

5. The FA-AC based nanocomposite of any one of claims 1 to 4, wherein the drug is an antibody therapeutic agent.

6. A composition for drug delivery, comprising the folic acid-conjugated aminoclay (FA-AC) based nanocomposite according to any one of claims 1 to 5 and a pharmaceutically acceptable carrier.

7. The composition of claim 6, wherein the composition is administered by orally, injection, buccally, nasally, sublingually, pulmonary, or dermally.

8. A pharmaceutical composition for the prevention or treatment of inflammatory diseases or cancer, comprising: the folic acid-conjugated aminoclay (FA-AC) based nanocomposite according to any one of claims 1 to 5; and a pharmaceutically acceptable carrier.

9. The pharmaceutical composition of claim 8, wherein the inflammatory diseases is ulcerative colitis.

10. A pH-sensitive folic acid-conjugated aminoclay (FA-AC) based nanocomposite, wherein a pH-sensitive polymer is coated on the FA-AC based nanocomposite according to any one of claims 1 to 5.

11. The pH-sensitive FA-AC based nanocomposite of claim 10, wherein the pH-sensitive polymer is a poly(methacrylic acid-co-methyl acrylate) copolymer.

12. The pH-sensitive FA-AC based nanocomposite of claim 10 or 11, wherein the pH-sensitive polymer is Eudragit® S100.

13. A pharmaceutical composition for oral administration, comprising:
the pH-sensitive folic acid-conjugated aminoclay (FA-AC) based nanocomposite according to any one of claims 10 to 12; and a pharmaceutically acceptable carrier.

14. A method of preparing a folic acid-conjugated aminoclay (FA-AC) based nanocomposite, the method comprising
(a) binding folic acid and aminoclay to each other to obtain FA-AC; and
(b) binding a drug to the FA-AC to obtain a FA-AC based nanocomposite.

15. The method of claim 14, wherein aminoclay is a metal phyllosilicate into which a 3-aminopropyl group is introduced.

16. The method of claim 14 or 15, wherein the drug is a protein drug, a peptide drug, DNA, RNA, an antibody therapeutic agent, an immunotherapy agent, or a small molecule drug.

17. The method of any one of claims 14 to 16, further comprising (c) coating a pH-sensitive polymer on the FA-AC based nanocomposite.

18. The method of any one of claims 14 to 17, wherein the pH-sensitive polymer is a poly(methacrylic acid-co-methyl acrylate) copolymer.

19. A method for the prevention or treatment of inflammatory diseases or cancer, the method comprising administering the folic acid-conjugated aminoclay (FA-AC) based nanocomposite according to any one of claims 1 to 5 to a subject.

20. Use of the folic acid-conjugated aminoclay (FA-AC) based nanocomposite according to any one of claims 1 to 5, for the prevention or treatment of inflammatory diseases or cancer.
